(19) United States
(12) Patent Application Publication
(10) Pub. No.: US 2018/0303663 A1
(43) Pub. Date: Oct. 25, 2018

(54) BALLOON CATHETER FOR ENDOVASCULAR TEMPERATURE CONTROL

(71) Applicant: Acandis GmbH & Co. KG, Pfinztal (DE)

(72) Inventors: Giorgio CATTANEO, Karlsruhe (DE); Michael BÜCHERT, Bretten (DE)

(21) Appl. No.: 15/767,933

(22) PCT Filed: Oct. 12, 2016
(86) PCT No.: PCT/EP2016/074498

§ 371 Date: Apr. 12, 2018

(30) Foreign Application Priority Data
Oct. 12, 2015 (DE) 10 2015 117 340.9

(51) Int. Cl. A61F 7/12; A61M 25/10

(52) U.S. Cl. CPC A61F 7/123; A61F 2007/0056; A61M 25/1011

(57) ABSTRACT

A balloon catheter for the endovascular temperature control of blood, with a catheter tube and at least one heat exchanger balloon which is convertible from an expanded operational state into a compressed insertion state. A temperature-control fluid can flow through the heat exchanger balloon, wherein the catheter tube includes at least one occlusion balloon which is arranged in series with the heat exchanger balloon.

The invention relates to a balloon catheter for the endovascular temperature control of blood in accordance with the preamble of patent claim 1. A balloon catheter of this type is known from EP 1 915 943 A1, for example.

EP 1 915 943 A1 discloses a balloon catheter which comprises a catheter tube on which a heat exchanger balloon is arranged. The heat exchanger balloon can be expanded by supplying fluid, whereupon an enlarged heat exchange surface area can be produced. The fluid is supplied via a supply lumen and a return lumen, which are formed in the catheter tube. In use, a coolant, for example cooled saline solution, flows through the heat exchanger balloon.

The function of the heat exchanger balloon is to cool blood which flows by it efficiently. This produces localized hypothermia, which means that metabolic processes are slowed down in the cooled regions of tissue. In this manner, endovascular treatments can be carried out with reduced time constraints. This reduces consequential damage, for example after a stroke or heart attack, and increases the chances of survival.

The usual cause of a stroke or heart attack is a blood clot (thrombus) which narrows or closes off the flow of blood through a blood vessel to such an extent that the supply of oxygen to the downstream regions of tissue is reduced. Thus, therapies for a stroke or heart attack are aimed at removing the thrombus as quickly as possible. Mechanical removal of a thrombus is preferably carried out in a minimally invasive manner using a catheter which is connected to a suction device. The thrombus can be sucked (aspirated) into the catheter using the suction device. In this regard, usually, the blood vessel upstream of the thrombus is closed off by means of an occlusion balloon. This is to avoid the possibility of individual components of a thrombus being detached during aspiration of the thrombus and being washed with the flow of blood into smaller blood vessels and cause another narrowing of the vessel there. Furthermore, closing off the blood vessel by means of the occlusion balloon prevents blood flowing to it from being sucked up and thus no longer available to the circulation in the body.

Both endovascular hypothermia and also endovascular occlusion are thus advantageous for a good therapeutic outcome in the treatment of strokes or heart attacks. However, the heat exchanger balloons of known hypothermia catheters are not suitable for closing off blood vessels. On the one hand, heat exchanger balloons are generally produced as non-elastic, dilatable balloons known as non-compliant balloons. The dimensions of the heat exchanger balloon are thus defined for a specific fluid pressure. On the other hand, the usual dimensions of heat exchanger balloons are such that after expansion in a blood vessel, they specifically do not make a seal, so that blood flowing by can be efficiently cooled.

The objective of the invention is to provide a balloon catheter for endovascular temperature control of blood, which improves the treatment of thromboses, in particular in the context of a stroke or heart attack.

In accordance with the invention, this objective is achieved by means of the subject matter of patent claim 1.

Thus, the invention is based on providing a balloon catheter for endovascular temperature control of blood, having a catheter tube and at least one heat exchanger balloon, wherein the heat exchanger balloon is convertible from an expanded operational state into a compressed insertion state. A temperature-control fluid can flow through the heat exchanger balloon. The catheter tube also comprises at least one occlusion balloon which is arranged in series with the heat exchanger balloon.

The invention is based on the concept that, in addition to the heat exchanger balloon, one or more occlusion balloons may be arranged on the catheter tube. The heat exchanger balloon and the at least one occlusion balloon are preferably arranged in series on the catheter tube. In this manner, the advantages of both therapeutic measures, hypothermia on the one hand and thrombectomy, in particular by aspiration, on the other hand, can advantageously be used in combination. In particular, manipulation by the physician is facilitated, because both functions are combined in a single balloon catheter, and thus no changes of instruments are required. Thus, for example, the heat exchanger balloon can initially be deployed in order to introduce hypothermia in the treatment region and to slow down metabolic functions. Next, without changing the catheter, the occlusion balloon can be deployed in order to close off the blood vessel and to prevent thrombus components from being washed away. The catheter can also be connected to a suction device so that it is possible to aspirate a thrombus by means of the catheter.

In a preferred embodiment, the occlusion balloon is elastically dilatable to at least 150%, in particular at least 200%, in particular at least 300%, in particular at least 400%, in particular at least 500% of its diameter in the non-operational state. The occlusion balloon in this regard can thus be dilated to an extent such that no damage or lack of tightness occurs. Preferably, the occlusion balloon is generally formed as a compliant balloon. The occlusion balloon is thus elastically dilatable so that the balloon fits well to the inner contour of a blood vessel and thus can efficiently seal it. A high dilatability is of particular advantage for deployment in larger blood vessels, for example the internal carotid artery (arteria carotis interna).

Preferably, in the non-operational state, the occlusion balloon lies tubularly against the catheter tube. In the non-operational state, then, the occlusion balloon has a comparatively small diameter. This means that the catheter can readily be inserted into narrow blood vessels.

The heat exchanger balloon is preferably formed as a non-compliant balloon. A certain amount of elasticity is not entirely excluded, however, compared with the elasticity of the compliant balloon or occlusion balloon, it is negligible. In this regard, the elasticity of the heat exchanger balloon is considered to be negligible, and thus the heat exchanger balloon is considered to be a non-compliant balloon when the heat exchanger balloon is elastically dilatable to at most 150%, in particular at most 130%, in particular at most 120%, in particular at most 110%, in particular at most 105% of its nominal diameter. As an example, the maximum diameter of the heat exchanger balloon for a nominal diameter of 4 mm and a dilation of 105% is 4.2 mm. The values provided above are valid for a relative pressure between the interior of the heat exchanger balloon and the environment of at most 500 mbar, in particular at most 400 mbar, in particular at most 300 mbar, in particular at most 200 mbar, in particular at most 100 mbar.

Upon dilation of the heat exchanger balloon beyond the degree of dilation mentioned above, the integrity of the heat exchanger balloon or the connection of the heat exchanger balloon with the catheter can no longer be guaranteed. In other words, the heat exchanger balloon is preferably configured such that it can withstand a dilation in the range of the degree of dilation mentioned above without damage. In particular, the maximum degrees of dilation mentioned above may be with respect to an absolute pressure inside the balloon of 2 bar, whereupon the balloon remains undamaged. In other words, the maximum degrees of dilation given here are obtained for an absolute pressure inside the balloon of 2 bar, wherein the balloon is in an environment at atmospheric pressure.

The nominal diameter of the heat exchanger balloon in preferred embodiments may be in the range 3 mm to 5 mm, in particular 4 mm. In this manner, the heat exchanger balloon is particularly suitable for deployment in the common carotid artery (arteria carotis comunis).

The nominal diameter corresponds to the diameter at which the heat exchanger balloon is at maximum dilation without any elastic deformation occurring. An elastic deformation of the heat exchanger balloon leads to an increase in the diameter and could result in an occlusion which, in contrast to the intended occlusion by the compliance balloon, which is only maintained during the brief thrombus aspiration phase, is maintained during the entire cooling period and thus hinders cooling of the blood and compromises the safety of the procedure. This is usually undesirable, and so an elastic dilation of the heat exchanger balloon should be avoided as far as possible, or in any case reduced to a negligible minimum.

In a further preferred embodiment of the catheter in accordance with the invention, the heat exchanger balloon is in fluid communication with a supply lumen and a return lumen and forms a closed temperature-control circuit. This allows for continuous temperature control, in particular cooling, of the blood flowing around the heat exchanger balloon.

In the context of the present invention, it is also possible for a plurality of heat exchanger balloons to be arranged in series on the catheter tube. The surface area for heat exchange can be increased by using a plurality of heat exchanger balloons, and thus the efficiency of the temperature control can be enhanced. The coolant may flow in succession through the plurality of heat exchanger balloons. As an example, the coolant could flow from the supply lumen initially into the distal heat exchanger balloon, then in sequence via all of the heat exchanger balloons to the proximal heat exchanger balloon, and then back into the return lumen. It is also possible for the flow through the heat exchanger balloons to be in the proximal to the distal direction.

In the context of the present invention, the term “proximal” is used to describe a region of the catheter which is arranged closest to the user or physician. Regions of the catheter which are arranged closer to the treatment site than to the user or physician are described as “distal” regions.

In this respect, in preferred embodiments, the occlusion balloon is preferably arranged distally from at least one heat exchanger balloon. In this case, the occlusion balloon is thus arranged closer to the treatment site than the heat exchanger balloon.

Alternatively, the occlusion balloon may be arranged proximally from at least one heat exchanger balloon. Here, the heat exchanger balloon is arranged closer to the treatment site than the occlusion balloon. The proximal disposition of the occlusion balloon has the advantage that temperature control can even be carried out by the distally arranged heat exchanger balloon when the blood vessel is completely closed off by the occlusion balloon. The heat exchanger balloon can then control, and in particular cool, the temperature of passive, static blood. This has the effect that after deflation of the occlusion balloon, substantially cooled blood flows distally and those areas of tissue which were under-supplied with nutrients because the vessel was sealed off, in particular the penumbral tissues, initially come into contact with substantially cooled blood. In this manner, particularly effective hypothermia is obtained in these particularly strongly compromised regions of tissue.

Placing the occlusion balloon distally to the plurality of heat exchanger balloons is of great advantage. In order to treat intracranial diseases, the at least one heat exchanger balloon is preferably placed in the arteria carotis communis in order to cool the blood in the proximal, collateral vessels, for example the vessels branching off from the arteria carotis externa. The occlusion balloon may be positioned in the arteria carotis interna so that during aspiration, blood is not aspirated from the arteria carotis externa. Disposing the occlusion balloon distally to the heat exchanger is particularly apposite for the aspiration of thrombi from the arteria carotis interna or vessels branching therefrom, for example the medial carotid artery (arteria carotis media). In addition to or as an alternative to aspiration, a mechanical instrument, for example a clot retriever, may be employed in order to remove thrombi.

It is possible for the single occlusion balloon to be positioned distally with respect to all of the heat exchanger balloons. As an alternative, the occlusion balloon may also be positioned proximally to all of the heat exchanger balloons. Furthermore, it is possible for the occlusion balloon to be arranged between two heat exchanger balloons. When a plurality of occlusion balloons are present, all of the occlusion balloons may be positioned distally or proximally with respect to all of the heat exchanger balloons. As an alternative, at least one occlusion balloon may be positioned proximally and at least one occlusion balloon may be positioned distally with respect to all of the heat exchanger balloons. One or more occlusion balls may be placed between heat exchanger balloons. Further heat exchanger balloons may also be located distally and/or proximally with respect to all of the heat exchanger balloons. In general, any disposition of occlusion balloons and heat exchanger balloons is possible along the catheter tube, irrespective of whether a plurality of occlusion balloons and/or heat exchanger balloons are provided or respectively only a single occlusion balloon and/or heat exchanger balloon is provided.

It is possible for the catheter tube to be provided with a proximal section and a distal section, wherein the proximal section is provided with a supply lumen and a return lumen which is in fluid communication with at least one heat exchanger balloon. The distal section may comprise one, in particular a single lumen for the occlusion balloon which is in fluid communication with the occlusion balloon. In other words, the distal section may be formed without a supply lumen and a return lumen. The supply lumen, the return lumen and the occlusion-fluid lumen preferably extend through the proximal section of the catheter tube. A through-lumen preferably extends through the catheter tube, i.e. through the proximal and the distal section. The flexibility of the catheter tube is increased by reducing the number of lumens in the distal section. A catheter tube of this type is particularly suitable for the application of the catheter as described above for the treatment of intracranial diseases, in particular for the removal of thrombi or for vessel recanalization.

In general, the proximal section may have a larger external diameter than the distal section. The proximal and the distal sections may each have a constant external diameter over their entire length. However, it is also possible for the proximal section and/or the distal section, in particular the entire catheter tube, to have an external diameter which changes in the longitudinal direction, in particular which tapers in the distal direction. As an example, the proximal section may have a distal tapering region which preferably adjoins the distal section. The heat exchanger balloons are advantageously arranged in the tapered region of the proximal section. There are preferably the same number of lumens in the tapered region of thermal proximal section as there are in a proximal region of the proximal section. In total, the catheter tube may consequently have three different external diameters, specifically a first external diameter in the proximal region of the proximal section, a second external diameter in the distal region of the proximal section and a third external diameter in the distal section, wherein the second external diameter is smaller than the first external diameter and larger than the third external diameter.

The transition between the proximal and distal sections and/or the transition between the adjoining regions within a section, for example between the proximal and distal region of the proximal section, may be seamless, in particular continuous. Preferably, the transition is smooth or without an abrupt transition.

In a further preferred embodiment of the invention, the occlusion balloon is in fluid communication with a lumen with a diameter of at most 1 mm, in particular at most 0.8 mm, in particular at most 0.6 mm, in particular at most 0.4 mm, in particular at most 0.3 mm, in particular at most 0.25 mm. Preferably, the occlusion balloon is in fluid communication with a single lumen. This reduces the complexity of the catheter and aids miniaturization. The lumen serves to introduce fluid into the occlusion balloon in order to expand it, in particular to dilate it elastically.

Furthermore, the catheter may comprise at least two lumens for the heat exchanger balloon, at least one lumen for the occlusion balloon and at least one further lumen for inserting a functional element, in particular a microcatheter, an intermediate catheter, a recanalization device or a thrombectomy device. The further lumen for the insertion of a functional element may in particular have a diameter which is suited for or compatible with passing through a catheter tube of the size 5 French or 6 French.

The further lumen is preferably configured as a through-lumen. In particular, the through-lumen is suitable for or designed for the introduction of treatment catheters, for example aspiration catheters, or of medical instruments, for example recanalization instruments such as clot retrievers.

Particularly preferably, the heat exchanger is in fluid communication with two lumens, for example a supply lumen and a return lumen, in order to produce a closed temperature-control circuit. The occlusion balloon is preferably in fluid communication with a single lumen in order to carry out a dilation or dilation of the occlusion balloon. Thus, together with the through-lumen for a functional element, the catheter preferably has four lumens. In particular, the lateral may have four lumens in a proximal section. In contrast, a distal section of the catheter which carries the occlusion balloon may only have two lumens, namely the through-lumen and the lumen for the occlusion balloon.

In general, the catheter has at least one lumen for supplying and/or withdrawing coolant, which may be formed as a supply lumen or return lumen or combined supply-and-return lumen. Preferably, the at least one lumen for supplying and/or withdrawing coolant extends only through the proximal section of the catheter tube. The distal section of the catheter tube is free from one lumen for supplying and/or withdrawing coolant. The distal section thus has at least one, in particular two lumens fewer than the proximal section.

Specifically, the proximal section of the catheter tube may have at least three lumens, preferably at least four lumens, in particular precisely four lumens. The distal section of the catheter tube preferably has at least two lumens, in particular precisely two lumens. Specifically, the proximal section of the catheter tube may have at least one more, in particular precisely two lumens more than the distal section of the catheter tube.

More specifically, the individual lumens do not have to extend completely through the proximal or distal section. Moreover, the lumens may extend only in regions through the respective section, i.e. the proximal section or the distal section. In this regard, the lumens may be interrupted within the proximal or distal section or discharge at a lateral opening of the catheter tube.

In general, the catheter tube may be equipped with further lumens, in particular for supplying and administering medication, contrast agents or other cold or warm fluids. The further lumens may extend through the entire catheter tube or may have an outlet opening, preferably a lateral outlet opening, at any point, in particular in the proximal section or in the distal section of the catheter tube.

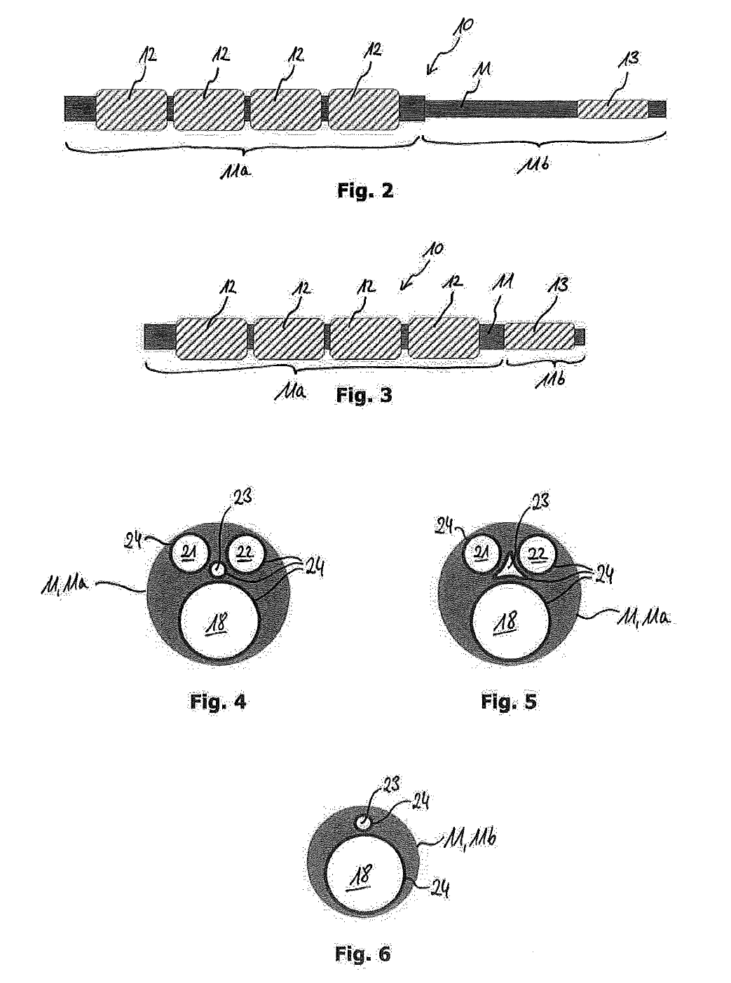

In a preferred embodiment of the invention, the supply lumen and the return lumen may respectively be kidney-shaped or in the shape of pulmonary lobes. On the other hand, the through-lumen may have a circular cross-section. Preferably, the through-lumen is arranged eccentrically, i.e. radially offset with respect to the central longitudinal axis of the catheter. Such a disposition and geometrical shape for the lumen is known, for example, from DE 10 2013 104 948 A1, the disclosure of which, in particular as regards paragraphs [0049] and [0050] as well as the drawings, FIG. 2, is incorporated in its entirety into the present application.

Furthermore, the lumen for the occlusion balloon or compliance balloon may have a circular cross-section. The lumen for the occlusion balloon may be formed between the return lumen and the supply lumen, in particular in a separating wall which separates the return lumen and the supply lumen. It is also possible for the lumen for the occlusion balloon to have a different cross-sectional shape, in particular a triangular cross-sectional shape, in order to make good use of the space between the supply lumen and the return lumen. The lumen for the occlusion balloon may be arranged in the centre in the separating wall. It is also possible for the lumen for the occlusion balloon to be offset. As an example, the lumen for the occlusion balloon may be offset in the direction of the outer wall of the catheter or in the direction of the through-lumen. The position in the separating wall is advantageous because the flow rate in the return lumen and the supply lumen is then barely influenced by the coolant. The lumen for the occlusion balloon may also be arranged in other positions, for example in the region of a tip of a kidney-shaped or pulmonary lobe-shaped lumen. In this manner, the surface area of the return lumen or the supply lumen for flow is indeed reduced, but not substantially compromised.

It is also possible for the at least four lumens, in particular in the proximal section of the catheter tube, to each have a round or circular cross-section. In this case too, the occlusion-fluid lumen may be arranged between the supply lumen and the return lumen. Preferably, the occlusion-fluid lumen is arranged in the space remaining between the supply lumen, the return lumen and the through-lumen. Alternatively, the occlusion-fluid lumen may in particular be positioned asymmetrically between the supply lumen or the return lumen and the outer circumferential surface of the catheter tube.

The individual lumens inside the catheter tube may respectively be surrounded by an essentially tubular material which differs from the material of the catheter tube. In particular, the catheter tube may be produced from a plastic matrix into which the tubular material is embedded to define the lumen. In other words, each lumen may be reinforced by its own inner tube. The inner tube may extend over the entire length of the lumen or reinforce the lumen in sections. The material of the inner tube preferably differs from the material of the plastic matrix of the catheter tube. Reinforcing individual lumens of the catheter tube increases the stability of the respective lumen against the fluid pressure in the lumen itself. In addition, the stability of the lumen against the fluid pressure in adjacent lumens is increased. In total, reinforcement by an inner tube in one or more lumens guarantees a good throughput through the individual lumens, in particular through the supply and/or return lumens and/or through the lumen for the occlusion balloon (occlusion-fluid lumen). In addition, the reinforcement of individual or more lumens by an inner tube ensures that the through-lumen maintains a stable internal diameter, so that instruments and/or treatment catheters can be reliably passed through the through-lumen.

One or more lumens, in particular the through-lumen, may also be reinforced with metal. As an example, a wire braid or a heically wound wire, also known as a coil, may be embedded in the inner tube of the respective lumen, in particular the through-lumen. The wire may have a round or a square cross-sectional profile and/or be formed from metal or plastic. Reinforcement of the respective lumens, in particular the through-lumen, by a wire means that the wall thicknesses between the individual lumens can be reduced, and thus a compact external diameter can be produced. Simultaneously, the respective lumens, in particular the through-lumen, can withstand high pressures which are concomitant with simultaneous filling of the occlusion balloon and the heat exchanger balloon. By means of the metal reinforcement, the through-lumen in particular can tolerate small bending radii without the cross-section of the through-lumen being deformed too greatly (ovalization).

In addition, in the context of the present invention, a system is disclosed with a balloon catheter as hereinbefore disclosed and claimed, wherein the system furthermore comprises an extracorporal cooling unit and a tubing set for connecting the catheter with the extracorporal cooling unit. The catheter preferably comprises a supply lumen and a return lumen which are connected to the tubing set on the one hand and to the at least one heat exchanger balloon on the other hand such that a closed coolant circuit is formable or is formed.

The system in accordance with the invention may advantageously be combined with or combinable with a treatment catheter, in particular with a supply catheter and/or an aspiration catheter, wherein the treatment catheter can be guided through a through-lumen of the catheter to the treatment site. Furthermore, the system may comprise a recanalization device, for example a clot retriever, which can be introduced into a blood vessel through the treatment catheter. The recanalization device may be guided to the treatment site via a microcatheter. The microcatheter may be inserted via the treatment catheter, or in fact directly via the through-lumen. Additionally or as an alternative, the treatment catheter may be connectable to a suction device or aspiration device so that the treatment catheter forms an aspiration catheter.

The system with the combination of the balloon catheter in accordance with the invention with the treatment catheters and/or devices mentioned above and/or the tubing set and/or the cooling unit mentioned above forms part of the invention and is disclosed in combination with all of the constructional features cited above.

The cooling unit may comprise at least one temperature control element for cooling a coolant flowing through the tubing set and at least one fluid delivery device for generating a flow of coolant inside the tubing set. Particularly preferably, the fluid delivery device is a peristaltic pump. In this manner, delivery of the coolant is possible under sterile conditions, since no parts of the pump come into direct contact with the coolant. The peristaltic pump is preferably designed such that it can produce a pressure of at least 3 bar for a coolant flow rate of 120 mL/min. A peristaltic pump designed in this manner is advantageous because the broader lumen (occlusion-fluid lumen) for the occlusion balloon means that space inside the catheter tube for the supply lumen and/or the return lumen is reduced, which results in a high flow resistance in the supply or return lumen.

With the proposed design thereof, the peristaltic pump can overcome this flow resistance.

The temperature control element may be formed by a Peltier element. In particular, two temperature control elements or Peltier elements may be provided which are arranged essentially parallel to each other, and between them is placed a receiving gap for a flow-through pouch for the coolant. Specifically, the tubing set may be provided with a flow-through pouch which can be inserted between two temperature control elements such that the coolant flowing through the pouch is cooled by the temperature control elements. The Peltier elements preferably have a cooling surface area of at least 150 cm², in particular 200 cm².

The invention will now be explained in more detail with the aid of exemplary embodiments, with reference to the accompanying diagrammatic drawings in which:

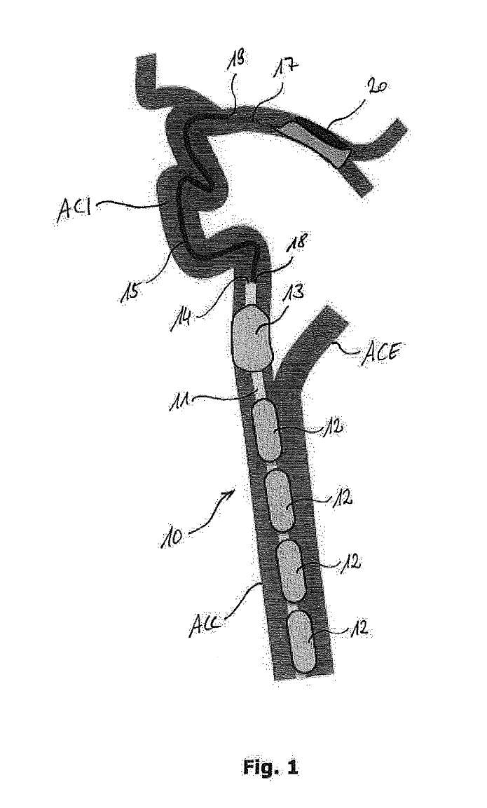

FIG. 1 shows a side view of a balloon catheter in accordance with the invention in a preferred exemplary embodiment in use in the treatment of a thrombus in the internal carotid artery;

FIGS. 2 and 3 each show a side view of a balloon catheter in accordance with the invention in a further preferred exemplary embodiment with a proximal section and a distal section of the catheter tube, which are configured in different manners;

FIGS. 4 and 5 each show a cross-sectional view through a proximal section of the catheter tube of a balloon catheter in accordance with the invention in accordance with a preferred exemplary embodiment; and

FIG. 6 shows a cross-sectional view through a distal section of the catheter tube of a balloon catheter in accordance with the invention in accordance with a preferred exemplary embodiment.

The exemplary embodiment depicted in FIG. 1 shows a balloon catheter 10 in use during the removal of a thrombus 20. The balloon catheter 10 is introduced into a blood vessel, specifically the internal carotid artery or arteria carotis interna, ACI. In general, the balloon catheter 10 is of particular application for the treatment of vessel blockages or clot formations in the region of the carotid artery.

The carotid artery comprises a main vessel, the arteria carotis communis, ACC, which divides into the arteria carotis interna, ACI and the external carotid artery or arteria carotis externa, ACE. In the exemplary embodiment depicted, a thrombus 20 has formed in the medial carotid artery, distal to the arteria carotis interna, ACI; the thrombus hinders the flow of blood into portions of the brain tissue. In order to remove the thrombus 20, the balloon catheter 10 in accordance with the invention can be used.

The balloon catheter 10 comprises a catheter tube 11 on which an occlusion balloon 13 is arranged. The occlusion balloon 13 is arranged at a distal section of the balloon catheter 10.

A plurality of heat exchanger balloons 12 are arranged on the catheter tube 11 proximally to the occlusion balloon 13. Four heat exchanger balloons 12 can be seen in the drawing. A different number of heat exchanger balloons 12 may also be envisaged.

In the depicted treatment status of the balloon catheter 10, the occlusion balloon 13 has been expanded and elastically dilated so that the occlusion balloon 13 seals against the vessel walls of the arteria carotis interna, ACI. In this regard, the occlusion balloon 13 is formed as a compliant balloon which is elastically dilatable beyond its nominal diameter. This guarantees a good seal with the blood vessel.

In contrast, the heat exchanger balloons 12 are formed as non-compliant balloons and have a nominal diameter which is preferably smaller than the nominal diameter of the occlusion balloon 13, in particular smaller than the diameter of the occlusion balloon 13 in use, i.e. when sealing a blood vessel. The heat exchanger balloons 12 essentially have no or only a negligible elastic dilatability. In particular, the dimensions of the heat exchanger balloons 12 are preferably such that they can be expanded to a diameter which is smaller than the vessel diameter. This ensures that blood can still flow by the heat exchanger balloons 12 and exchange heat with the blood which is flowing by.

In the exemplary embodiment shown, the balloon catheter 10 comprises a through-lumen 18 which accommodates a guide catheter 15, for example. The guide catheter 15 here is guided inside the through-lumen 18 in a longitudinally displaceable manner and can leave the through-lumen at a distal opening 14. In addition to guiding the guide catheter 15, the through-lumen may also be used for aspiration. In this manner, blood and, if appropriate, detached thrombus components can be sucked away.

The guide catheter 15 can be pushed up to close to the thrombus 20. In particular, the guide catheter 15 is highly flexible so that it can be guided correctly through narrow and tortuous blood vessels to the treatment site. The guide catheter 15 comprises a through channel 19 through which a microcatheter 17 can be pushed. In preferred embodiments of the invention, the guide catheter 15 can be connected to suction test equipment so that aspiration can be carried out via the guide catheter 15. In this manner, components of the thrombus close to the treatment site can be sucked away. In addition, the through-lumen 18 of the catheter tube 11 may be connected to or connectable to a suction device or aspiration device.

Preferably, a longitudinally displaceable transport wire is arranged inside the microcatheter 17 which is firmly attached to or releasably attached to a thrombectomy device 16 at a distal end. The thrombectomy device 16 can be pushed onto the thrombus 20 by means of the transport wire. As an example, the thrombectomy device 16 might be a self-expandable lattice structure which connects itself to the thrombus 20. In this manner, the thrombus 20 can be removed with the aid of the thrombectomy device 16 and be withdrawn into the catheter tube 11.

Further lumens with different functions may extend in the catheter tube 11 in addition to the through-lumen 18. Thus, at least one lumen is provided which is in fluid communication with the occlusion balloon 13. The occlusion balloon 13 can be expanded and compressed again via the lumen which is connected to the occlusion balloon 13. To this end, a fluid, for example saline solution, preferably with added contrast agents, is fed into the occlusion balloon 13 or withdrawn therefrom.

The heat exchanger balloons 12 are preferably connected to two lumens, wherein the heat exchanger balloons 12 have a supply lumen 21 on one side and a return lumen 22 on the other side. The fluid connections between the individual heat exchanger balloons 12 and the supply lumen 21 or the return lumen 22 are preferably arranged at the respective longitudinal ends of the heat exchanger balloon 12. In particular, the supply lumen 21 may discharge at a distal end of the heat exchanger balloon 12 and the return lumen 22 may be in fluid communication with a proximal end of the heat exchanger balloon 12. This ensures that temperature-control fluid which reaches the heat exchanger balloon 12 via the supply lumen 21 flows through the entire heat exchanger balloon 12 before it is withdrawn from the heat exchanger balloon 12 via the return lumen 22. It is also possible to envisage each heat exchanger balloon 12 having a respective supply lumen 21 and a return lumen 22. However, preferably, the heat exchanger balloons 12 are connected together with a single supply lumen 21 and a single return lumen 22. In this regard, the heat exchanger balloons 12 are preferably connected in series or belong to a common temperature-control circuit. In particular, it is possible for a single supply lumen 21 to be connected to the distal or proximal heat exchanger balloon 12 and for the heat exchanger balloons 12 to be connected together in series via the return lumen 22.

The occlusion balloon 13 is preferably designed such that the diameter of the occlusion balloon 13 increases by 1 mm above its non-operational state at a fluid pressure of less than 0.5 bar. In particular, the occlusion balloon 13 may be designed such that its diameter enlarges by 3 mm beyond the non-operational state when a pressure of less than 1 bar prevails inside the occlusion balloon 13. In the non-operational state, the occlusion balloon 13 is preferably tubular in shape, wherein the internal diameter of the occlusion balloon 13 essentially corresponds to the external diameter of the catheter tube 11. Preferably, the diameter of the occlusion balloon 13 in the non-operational state is at least 0.4 mm, 0.6 mm, 0.8 mm, 1.0 mm, 1.2 mm, 1.4 mm, 1.6 mm, 1.8 mm, 2.0 mm smaller than the diameter of the heat exchanger balloon 12 or of the plurality of heat exchanger balloons 12. This is the case for all of the exemplary embodiments of the invention.

In contrast, the heat exchanger balloons 12 are configured such that at a pressure of at least 1 bar which is applied by the temperature-control fluid to the heat exchanger balloon 12, it enlarges in diameter by less than 0.5 mm compared with the non-operational state diameter. In this regard, the non-operational state essentially corresponds to a state in which the heat exchanger balloon 12 lies almost completely against the catheter tube 11. The nominal diameter of the heat exchanger balloon 12 is reached when an elastic deformation occurs upon increasing the pressure inside the heat exchanger balloon 12 further. This elastic deformation is preferably limited such that the elastic dilation of the heat exchanger balloon 12 with respect to the nominal diameter is a maximum of 20%, in particular a maximum of 10%, in particular a maximum of 5%. Here again, this is the case for all of the exemplary embodiments of the invention. The values mentioned above are in particular valid for a pressure of 2 bar.

Regarding the use of the balloon catheter in accordance with the invention, several possibilities may be envisaged. On the one hand, the balloon catheter 10 may be used for the removal of thrombi 20.

On the other hand, it is also possible for the occlusion balloon 13 to be used for vessel dilation or for stenosis dilation. In this case, the heat exchanger balloon 12 or the occlusion balloon 13 may also undertake the function of blocking stenosis particles which are sometimes detached during dilation. For such an application, it may be advantageous to provide an additional aspiration lumen which discharges between the occlusion balloon 13 and the heat exchanger balloon 12. In this manner, blood and particles can efficiently be sucked up from the region between the occlusion balloon 13 and the heat exchanger balloon 12.

FIG. 2 shows a further exemplary embodiment of the balloon catheter 10 in accordance with the invention. The particular feature of this exemplary embodiment is that the catheter tube 11 of the balloon catheter 10 has a proximal section 11a and a distal section 11b. The occlusion balloon 13 is arranged in the distal section 11b. The proximal section 11a carries a plurality of heat exchanger balloons 12. The proximal section 11a and the distal section 11b differ in particular in their external diameters. In particular, the distal section 11b of the catheter tube 11 has a smaller cross-sectional diameter than the proximal section 11a.

The different cross-sectional diameters result from the internal construction of the catheter tube 11, which differ in the distal section 11b and in the proximal section 11a. A through-lumen 18 extends through the entire catheter tube 11. The through-lumen 18 may be used to supply medication, cold or warm fluids such as contrast agents, treatment catheters or instruments, in particular to suck up or aspirate or remove blood clot particles. Furthermore, a supply lumen 21 and a return lumen 22 as well as a lumen for the occlusion balloon, in particular an occlusion-fluid lumen 23, extend through the catheter tube 11. The supply lumen 21 and the return lumen 22 are in fluid communication with the heat exchanger balloons 12, so that a coolant circuit can be formed. In this regard, the supply lumen 21 and the return lumen 22 may be connected to an extracorporal cooling unit via a tubing set so that a coolant circuit is produced which provides for a continuous supply of coolant and withdrawal of coolant for the heat exchanger balloons 12.

The supply lumen 21 and the return lumen 22 preferably extend only in the proximal section 11a of the catheter tube 11. In addition, the occlusion-fluid lumen 23 and the through-lumen 18 extend through the proximal section 11a. In all of the exemplary embodiments of the invention, the through-lumen 18 also extends through the distal section 11b. The occlusion-fluid lumen 23 may also extend through the distal section 11b or merge directly into the occlusion balloon 13 at the distal end of the proximal section 11a. Such an exemplary embodiment is shown in FIG. 3. In particular, the front of the occlusion-fluid lumen 23 can merge into the occlusion balloon 13 at the distal end of the proximal section.

When the occlusion balloon 13 is arranged distally from the distal end of the proximal section 11a of the catheter tube 11, then the occlusion-fluid lumen 23 also flows inside the distal section. A configuration of this type is shown in FIG. 2.

Similarly, in preferred exemplary embodiments, the supply lumen 21 and the return lumen 22 are arranged only inside the proximal section 11a of the catheter tube 11. Because of the smaller number of lumens, the distal section 11b may thus be thinner, i.e. it may have a smaller external diameter.

FIGS. 4 and 5 respectively show different configurations of the proximal section 11a of the catheter tube 11 in cross-section. The proximal section 11a of the catheter tube has a through-lumen 18 which has a circular cross-section. Furthermore, two essentially identically sized coolant lumens are provided which each have a circular cross-section and form a supply lumen 21 and a return lumen 22. The supply lumen 21, the return lumen 22 and the through-lumen 18 are essentially arranged in a triangular arrangement. The occlusion-fluid lumen 23 is arranged between the supply lumen 21, the return lumen 22 and the through-lumen 18. The occlusion-fluid lumen 23 may have different cross-sectional profiles; in this regard, FIG. 4 shows an exemplary embodiment with an occlusion fluid 23 which has a circular cross-section. FIG. 5 shows an occlusion-fluid lumen 23 with an essentially triangular cross-section. This configuration uses the space between the remaining lumens 21, 22, 18 particularly effectively.

FIG. 6, on the other hand, shows a cross-section through a distal section 11b of the catheter tube 11. In particular, FIG. 6 can depict a section through the distal section 11b of the catheter tube 11 of FIG. 2, whereas FIG. 4 shows a section through the proximal section 11b of the catheter tube 11 of FIG. 2. In the distal section, the catheter tube 11 of FIG. 6 comprises only the through-lumen 18 and the occlusion-fluid lumen 23. Because the supply lumen 21 and the return lumen 22 have been dispensed with, the external diameter of the distal section 11b of the catheter tube 11 is reduced. In this manner, the manoeuvrability of the balloon catheter 10 is enhanced, in particular in small, intracranial vessels or vessels going to the brain.

The cross-sectional drawings of FIGS. 4 to 6 clearly show that the individual lumens (through-lumen 18, supply lumen 21, return lumen 22, occlusion-fluid lumen 23) are each formed with an inner tube 24. The inner tube 24 lines the individual lumens. Preferably, the inner tube 24 comprises a material which differs from the material of the catheter tube 11.

As an example, the inner tube 24 may consist of polyimide or polyamide or PTFE or a similar material. The inner tube 24 may be reinforced with a metal, for example in the form of a helically wound wire (coil). The metal preferably includes stainless steel or a nickel-titanium alloy, in particular Nitinol. The plastic matrix, i.e. the catheter tube 11 itself, is preferably formed from polyurethane or polyether block amide (PEBA) or polyamide (nylon) or polyethylene (PE) or Teflon. The heat exchanger balloons 12 may consist of polyamide or polyurethane or PEBA or PE. The catheter tube may be at least partially coated. In particular, a hydrophilic coating may be provided.

Furthermore, the through-lumen 18 or an inner tube 24 of the through-lumen 18 may be provided with a circumferential inner surface which is provided with a coating formed from PTFE or fluoroethylene propylene (FEP). The inner tube 24 itself may also consist of a friction-reducing material or may have an inner lining which faces the through-lumen and is friction-reducing. The friction-reducing material may include PTFE or FEP or consist of it. In addition, the inner tube 24 of the through-lumen 18 may be reinforced with metal.

The inner tube 24, in particular the inner tube 24 of the through-lumen 18, may be multi-layered. As an example, an inner layer facing the through-lumen 18 may be formed from a friction-reducing material, in particular PTFE or FEP, and an outer layer may be formed by polyurethane or Pebax. The outer layer may in general be formed from the same material as the plastic matrix of the catheter tube 11 or a material which differs therefrom.

The entire catheter tube 11, in particular also in the distal section 11b of the catheter tube 11, may have the cross-sectional construction of the through-lumen 18, in particular of the through-lumen 18 equipped with a multi-layered inner tube 24. In particular, the inner tube 24, for example with an inner layer formed from a friction-reducing material and an outer layer formed from another material as well as a metal reinforcement, may continue unchanged into the distal section 11b. The outer layer may be identical in the distal section 11b and in the proximal section 11a. However, it is also possible for the outer layer in the distal section 11b of the catheter tube 11 to consist of a material which is softer than the material of the outer layer in the proximal section 11a of the catheter tube 11. Similarly, the material of the plastic matrix of the catheter tube 11 in the distal section 11b may differ from the material of the plastic matrix of the catheter tube 11 in the proximal section 11a; in particular, the distal section 11b may be softer or more deformable than in the proximal section 11a.

Particularly preferably, the lumens, in particular the supply lumen 21, the return lumen 22 and/or the occlusion-fluid lumen 23 have an inner tube 24 or a circumferential inner surface of the respective lumen which consists of or is coated with polyimide.

For all exemplary embodiments with a catheter tube 11 which have a proximal section 11a and a distal section 11b, the external diameter of the catheter tube 11, at least in the proximal section 11a, may be in the range 3 mm to 4 mm, in particular in the range 3.2 mm to 3.8 mm, preferably in the range 3.4 mm to 3.6 mm.

The distal section 11b of the catheter tube 11 may exclusively comprise the through-lumen 18 and the occlusion-fluid lumen 23. The external diameter of the catheter tube 11 in the distal section 11b is preferably in the range 2 mm to 3.5 mm, in particular in the range 2.3 mm to 3.2 mm, in particular in the range 2.5 mm to 3 mm, preferably 2.8 mm.

The difference between the external diameter of the proximal section 11a and the external diameter of the distal section 11b is preferably at least 0.2 mm, in particular at least 0.4 mm, in particular at least 0.6 mm, in particular at least 0.8 mm, and/or at most 1.5 mm.

In a variation of the balloon catheter 10 in accordance with the invention, the length of the distal section 11b without the supply lumen 21 and return lumen 22 may be in the range 10 mm to 50 mm, in particular in the range 20 mm to 40 mm. These variations are suitable for a treatment in which, because of the short distance between the occlusion balloon 13 and the proximal section 11a, the risk of injury is to be reduced. In an alternative variation, which may be employed in order to seal off a vessel highly distally, i.e. at a a large distance from the proximal section 11a, the length of the distal section 11b may be in the range 20 mm to 150 mm, in particular in the range 30 mm to 120 mm, in particular in the range 40 mm to 100 mm, in particular in the range 50 mm to 80 mm. For both of these variations, the occlusion balloon 13 is preferably very close to or directly at the distal end of the distal section 11b, i.e. at the tip of the catheter tube 11. In particular, the distance of the occlusion balloon 13 or the distal end of the occlusion balloon 13 from the tip of the catheter tube 11 is at most 10 mm, in particular at most 8 mm, in particular at most 6 mm, in particular at most 4 mm. The distance between the occlusion balloon 13 and the tip of the catheter tube 11 may be at least 1 mm.

The occlusion balloon 13 preferably has a length in the range 3 mm to 20 mm, in particular in the range 5 mm to 15 mm, in particular in the range 8 mm to 12 mm. The wall thickness of the occlusion balloon 13 may be at most 100 μm, in particular at most 80 μm, in particular at most 60 μm, in particular at most 40 μm, in particular at most 20 μm. Preferably, the wall thickness is at least 10 μm. Suitable materials for the occlusion balloon 13 are Kraton and/or Chronoprene and/or Pellethane and/or latex and/or silicone.

The segment which contains the heat exchanger balloons 12, in particular the section of the catheter tube 11, which is defined proximally by the proximal end of the first heat exchanger balloon 12 and distally by the distal end of the last heat exchanger balloon 12, preferably has a length which is in the range 20 mm to 150 mm, in particular in the range 40 mm to 120 mm, in particular in the range 60 mm to 100 mm, preferably 80 mm. Each heat exchanger balloon 12 may have a respective length in the range 10 mm to 30 mm, in particular 20 mm. The wall thickness of the heat exchanger balloon 12 is preferably in the range 10 μm to 40 μm, in particular in the range 15 μm to 30 μm.

The occlusion balloon 13 may be funnel-shaped at its distal end or merge with the catheter tube 11 in the shape of a funnel. In this manner, introduction of the occlusion balloon 13 into a blood vessel, in particular into a section of a vessel with a thrombus 20, is thus facilitated. It is also possible for the occlusion balloon 13 to be in the shape of a funnel which widens in the distal direction, in order to thereby facilitate introduction of a thrombus 20 into the catheter 11 upon aspiration.

The occlusion balloon 13 may be in fluid communication with a plurality of occlusion-fluid lumens 23, or the occlusion-fluid lumen 23 may be divided into a plurality of part lumens which are each arranged in the space left between the supply lumen 21, the return lumen 22 and the through-lumen in order to exploit the available space inside the catheter tube 11 to the best extent possible. The balloon catheter 10 may be provided with a plurality of, in particular two occlusion balloons 13, so that at the same time an occlusion, i.e. closing off of a vessel, can be carried out at different locations.

Moreover, a radiographic marker may be provided at the proximal and distal ends of the occlusion balloon 13. This aids the user in correctly positioning the occlusion balloon 13 in the blood vessel. It is also possible to provide at least one radiographic marker at only one end of the occlusion balloon 13 and/or in the middle of the occlusion balloon 13. In addition to or as an alternative, a respective radiographic marker may be placed at the distal tip of the catheter tube 11 and/or at the proximal and/or distal end of the segment of the catheter tube 11 in which the heat exchanger balloons 12 are arranged. In total, then, three radiographic markers, two end radiographic markers at the proximal and distal ends of the occlusion balloon 13, as well as one in the middle of the occlusion balloon 13, may be provided.

The supply lumen 21 and the return lumen 22 may be closed at the distal end of the proximal section 11a of the catheter tube 11 by a melting process and/or a bonding process. In general, then, the supply lumen 21 and the return lumen 22 are closed at the distal end of the proximal section 11a of the catheter tube 11. The supply lumen 21 and the return lumen 22 may then be respectively connected to the heat exchanger balloons 12 via lateral, in particular radial openings. In analogous manner, the occlusion-fluid lumen 23 may be closed distally by bonding or melting and be connected to the occlusion balloon 12 by means of a lateral, in particular radial opening.

The supply lumen 21 and the return lumen 22 may respectively have an internal diameter in the range 0.5 mm to 1.5 mm, in particular in the range 0.8 mm to 1.2 mm, preferably 1 mm. In this manner, a sufficient volume flow of coolant is ensured. At the same time, the supply lumen 21 and the return lumen 22 take up only a little space in the catheter tube 11, so that sufficient space remains for a through-lumen 18 of suitable dimensions.

In general, the occlusion balloon 13 may be arranged at any location along the catheter tube 11. In particular, the occlusion balloon 13 may be arranged both in the proximal section 11a, and also in the distal section 11b of the catheter tube. In the proximal section 11a, the occlusion balloon 14 may be proximal or distal to the at least one heat exchanger balloon. When the occlusion balloon 13 is arranged in the proximal section 11a, the distal section 11b is free from balloons. In this case, the catheter tube 11 in the distal section 11b has only the through-lumen 18. The distal section 11b is then particularly flexible and inserting the balloon catheter 10 into narrow, tortuous blood vessels is facilitated.

At the proximal end of the balloon catheter 10, in particular of the catheter tube 11, separate connections may be provided for one or more of the lumens (through-lumen 18, supply lumen 21, return lumen 22, occlusion-fluid lumen 23).

The connections may be parts of a common Luer connector. It is also possible for each lumen to have its own Luer adapter or connector. In particular, each lumen may be assigned to a connection line which has a Luer adapter or connector at one proximal end.

The total length of the balloon catheter 10 or the catheter tube 11 is preferably in the range 40 cm to 150 cm. In particular, the total length may be in the range 70 cm to 120 cm, preferably in the range 80 to 100 cm, specifically cm. The values given just above are of particular advantage for a balloon catheter for neurovascular applications.

The distal end or the distal tip of the catheter tube 11, in particular of the distal section 11b of the catheter tube 11, is preferably rounded. This reduces the risk of injury when passing the balloon catheter 10 through blood vessels.

### LIST OF REFERENCE NUMERALS

10 balloon catheter
11 catheter tube
11a proximal section
11b distal section
12 heat exchanger balloon
13 occlusion balloon
14 distal opening
15 guide catheter
16 thrombectomy device
17 guide catheter
18 through-lumen
19 through channel
20 thrombus
21 supply lumen
22 return lumen
23 occlusion-fluid lumen
24 inner tube
ACI arteria carotis interna
ACE arteria carotis externa
ACC arteria carotis communis

1. A balloon catheter for the endovascular temperature control of blood, having a catheter tube and at least one heat exchanger balloon which is convertible from an expanded operational state into a compressed insertion state, wherein a temperature-control fluid can flow through the heat exchanger balloon, characterized in that the catheter tube comprises at least one occlusion balloon which is arranged in series with the heat exchanger balloon.

2. The catheter as claimed in claim 1, characterized in that the occlusion balloon is elastically dilatable to at least 150%, in particular at least 200%, in particular at least 300%, in particular at least 400%, in particular at least 500% of its diameter in the non-operational state.

3. The catheter as claimed in claim 1, characterized in that in the non-operational state, the occlusion balloon lies tubularly against the catheter tube.

4. The catheter as claimed in claim 1, characterized in that the heat exchanger balloon is elastically dilatable to at most 150%, in particular at most 130%, in particular at most 120%, in particular at most 110%, in particular at most 105% of its nominal diameter.

5. The catheter as claimed in claim 1, characterized in that the heat exchanger balloon is in fluid communication with a supply lumen and a return lumen and forms a closed temperature-control circuit.

6. The catheter as claimed in claim 1, characterized in that a plurality of heat exchanger balloons are arranged in series on the catheter tube.

7. The catheter as claimed in claim 1, characterized in that the occlusion balloon is arranged distally to at least one heat exchanger balloon.

8. The catheter as claimed in claim 1, characterized in that the occlusion balloon is arranged proximally to at least one heat exchanger balloon.

9. The catheter as claimed in claim 1, characterized in that the occlusion balloon is in fluid communication with a lumen with a diameter of at most 1 mm, in particular at most 0.8 mm, in particular at most 0.6 mm, in particular at most 0.4 mm, in particular at most 0.3 mm, in particular at most 0.25 mm.

10. The catheter as claimed in claim 1, characterized in that the catheter tube comprises at least two lumens for the heat exchanger balloon, at least one lumen for the occlusion balloon and at least one further lumen for inserting a functional element, in particular a microcatheter, an intermediate catheter, a recanalization device or a thrombectomy device.

11. A system having a catheter as claimed in claim 1, an extracorporal cooling unit and a tubing set for connecting the catheter to the extracorporal cooling unit, wherein the catheter tube comprises a supply lumen and a return lumen which are connected to the tubing set on the one hand and to the heat exchanger balloon on the other hand such that a closed coolant circuit is formable or formed.

12. The system as claimed in claim 11, characterized in that the cooling unit comprises at least one temperature-control element for cooling a coolant flowing through the tubing set and at least one fluid-delivery device for generating a flow of coolant inside the tubing set.

13. The system as claimed in claim 12, characterized in that the fluid-delivery device is a peristaltic pump.

14. The system as claimed in claim 12, characterized in that the temperature-control element is formed by a Peltier element.

15. The system as claimed in claim 12, characterized in that the tubing set is provided with a flow-through pouch which can be inserted between two temperature-control elements such that the coolant flowing through the pouch is cooled by the temperature-control elements.
